(19) United States
(12) Patent Application Publication
(10) Pub. No.: US 2015/0322493 A1
(43) Pub. Date: Nov. 12, 2015

(54) METHOD AND ITS COMPOSITIONS FOR DETECTION OF NUCLEIC ACID TARGET FROM BIOLOGICAL SAMPLES AND BODY FLUIDS

(71) Applicant: SELFDIAGNOSTICS OÜ, Harju County (EE)

(72) Inventors: Indrek TULP, Tartu (EE); Katrin KROLOV, Tartu (EE); Marko LEHES, Rae Parish (EE); Ulo LANGEL, Stockholm (SE)

(21) Appl. No.: 14/437,110

(22) PCT Filed: Oct. 20, 2013
(86) PCT No.: PCT/EP2013/071906

§ 371 Date: Apr. 20, 2015

Related U.S. Application Data
(60) Provisional application No. 61/716,485, filed on Oct. 20, 2012.

(51) Int. Cl. C12Q 1/68

(52) U.S. Cl. CPC C12Q 1/689; C12Q 2600/158

(57) ABSTRACT

Current invention is directed for rapid sample pretreatment method that allows highly sensitive and specific detection of target nucleic acid (eg human genomic DNA, human pathogen genomic DNA, human non-pathogen genomic DNA) by amplification directly from crude unpurified biological samples lysates (eg human urine, saliva, blood, urethra and cervical swabs and other samples containing biological material). Invention is focused on the description of the biological sample pretreatment method that enables fast release of the genomic material from human and pathogen cells, components of what are compatible with the following nucleic acid amplification method. As an example of the application, invention also discloses protocols and primer sequences for isothermal nucleic acid amplification (recombinase polymerase amplification—RPA, loop-mediated isothermal amplification—LAMP), that enable highly specific and sensitive diagnostics of the genomic material from Homo sapiens, Chlamydia trachomatis and Mycoplasma genitalium from crude biological sample lysates and/or purified total DNA. The example amplification can be combined with immunochromotographic product detection using lateral-flow strips and allows rapid (under 20 min) isothermal nucleic acid amplification based C. trachomatis and M. genitalium diagnostics from human urine samples, that does not require specific laboratory equipment nor qualified personnel, and is therefore well suited for point-of-care settings applications.

### PRIORITY

This application is a national entry of PCT/EP2013/071906 filed on Oct. 20, 2013 and claiming claims priority of U.S. 61/616,495 filed on Jun. 4, 2010, both of which are fully incorporated herein by reference.

### SEQUENCE LISTING

This application contains sequence data provided on a computer readable diskette and as a paper version. The paper version of the sequence data is identical to the data provided on the diskette.

### FIELD OF THE INVENTION

The invention is directed to compositions and method for rapid biological sample pretreatment that allows following nucleic acid amplification based detection of the target nucleic acid from biological samples and body fluids.

### BACKGROUND OF THE INVENTION

Current diagnostics relies majorly on the nucleic acid amplification techniques (NAAT). Most commonly known method for specific DNA amplification is PCR that gives reasonable sensitivity on the laboratory level. Lately new emerging techniques have been developed of isothermal amplification, such as recombinase polymerase amplification (RPA), loop-mediated isothermal amplification (LAMP), helicase dependent amplification (HDA). These isothermal NAATs do not require thrermocycling of the reaction and have shown extremely high levels of sensitivity, resulting in detectable amplification product from as few as 1-2 template copies. Isothermal reaction makes them well suited for point-of-care (POC) settings (eg GP office, at home), bringing diagnostics test conveniently and immediately to the patient and decreasing time to result. In the field of sexually transmitted diseases, POC diagnostics also allows private and non-invasive testing, that has a potential to significantly reduce the spread of the pathogens, especially those that exist in asymptomatic form like C. trachomatis and M. genitaium.

Both M. genitalium and C. trachomatis infections are known as “silent” diseases as they often remain asymptomatic. Thus regular diagnostic screening of these sexually transmitted pathogens is of high importance. Classically C. trachomatis infection has been diagnosed from urethral or cervical swab specimens by tissue culture method. Because culturing identifies only viable C. trachomatis cells, sensitivity of the diagnostics is affected by the freshness of the specimen depending on the time between collection and processing in the laboratory. Thus during 1980s antigen and nucleic acid detection technologies have been developed for C. trachomatis diagnostics that have lesser demand of cost, time, expertise, preservation of infectivity during transport. Furthermore nucleic acid detection techniques have proved to have much higher sensitivity levels as they can detect pathogen DNA from unviable cells or cell debris. Microbiological detection of M. genitalium is also mostly performed by specific amplification of the pathogen DNA by PCR. M. genitalium culture is extremely difficult and is not performed routinely. Serological detection methods of M. genitalium are weakly sensitive and specific.

Although NAAT open up crucial opportunity for highly effective diagnostics, to date they are routinely used only on the laboratory level. NAATs are complicated to perform, require trained personnel and expensive machinery. Thus NAAT based diagnostics is centered to large hospitals and diagnostics centers. One of the major limitations of the NAAT techniques is the requirement for pure DNA sample. The purity of the sample can affect significantly performance of the NAAT-s, especially PCR. Novel isothermal NAAT-s like RPA, LAMP, HDA etc seem to be less sensitive towards nucleic acid sample purity and are able to efficiency amplify DNA present in eg human urine samples.

Current invention discloses a method and its compounds for biological sample pretreatment that allows efficient release of the genomic DNA from cellular material. Described sample pretreatment method is compatible with the following nucleic acid amplification procedure allowing detection of the target DNA from crude sample lysates. The invention allows skipping of the DNA purification step prior to NAAT analysis, having therefore an important impact on the complexity and speed of the diagnostic technique. Current invention facilitates significantly implementation of the highly sensitive and specific NAAT diagnostics in the POC settings.

Because examples of the invention implementation is concentrated on human sexually transmitted pathogen diagnostics, the overview of the Chlamydia trachomatis and Mycoplasma genitalium will be given hereafter.

C. trachomatis and M. genitalium are sexually transmitted human pathogens. Both of them are associated with non-gonococcal (non-specific) urethritis in men and several inflammatory reproductive tract syndromes in women such as cervicitis and pelvic inflammatory disease. Inflammatory diseases caused by acute untreated infections of C. trachomatis and M. genitalium are one of the leading causes of female infertility worldwide.

The prevalence of M. genitalium ranges globally from 1-4% in men and 1-6% in women. Reported prevalence data within populations at higher risk (eg within sexually transmitted disease (STD) testing centers) reach 38%. C. trachomatis prevalence rates among sexually active young people vary from 5-10% depending on the age, ethnic origin etc. C. trachomatis infection is almost always more prevalent among women and has shown an increasing trend globally during past decades.

M. genitalium is a small (0.2-0.3 μm) pleomorphic bacterium that lacks cell wall making it resistant to common antibiotics targeting cell wall (eg penicillin). M. genitalium cells are flask shaped and carry a specific adhesion organelle that allows bacteria to adhere to various materials and cells including human epithelial cells. Adhesion is the main mechanism of M. genitalium pathogenesis that involves at least seven adhesins including major adhesin MgPa (encoded by MGPB gene).

C. trachomatis is a gram-negative, obligate intracellular pathogen that has a unique biphasic developmental cycle during which they exist in two developmental forms: the EB (or elementary body) and RB (or reticulate body). EB is smaller (0.2 μM), metabolically inactive, infectious extracellular form of the organism and RB is larger (0.8 μM) metabolically active intracellular form. Chlamydial infection involves attachment of the EB to a host cell and its subsequent internalization into a membrane-bound vesicle. Inclusion differentiates into RB which uses host cell ATP and metabolites to undergo 8-12 round of cell division. RB differentiates and matures into infectious EB that are released by host cell lysis. C. trachomatis strains are serologically classified into 15 serovars based on antigenic variation of the major outer membrane protein. A-C serovars are eye pathogens causing ocular trachoma. Serovars D-K and L1-L2 are sexually transmitted pathogens that infect columnar epithelial cells of the genital tract.

Adaptive immunity against C. trachomatis involves INF-γ mediated host cell responce that deprives chlamydial RBs of tryptophan, which ultimately prevents their growth and replicative capabilities. C. trachomatis genital serovars have retained some of the eubacterial tryptophan biosynthesis genes, TRPA and TRPB encoding α and β subunits of the tryptophan synthase that catalyzes conversion of the indole into tryptophan. Thus genital C. trachomatis serovars have retained the capacity to use exogenous indole secreted by genital trakt normal microflora that allows them to overcome INF-γ mediated growth restriction and promotes long term establishment of the infection.

M. genitalium has a small AT rich (68%) 0.58 Mb genome that encodes 485 genes. Despite its small size, 4% of the genome consists of repeated elements (MgPa repeats) that present homology with the MGPB gene. C. trachomatis also carries a small genome of approximately 1 Mb chromosome and 7.5 kb cryptic plasmid. Almost all C. trachomatis strains harbor four to ten plasmid copies per chromosome. Although some plasmid-free C. trachomatis isolates have been described, their virulence is significantly reduced as compared to the plasmid carrying strains. Chlamydia plasmid sequence is highly conserved (<1% variation) and contains eight major coding sequences (CDSs) along with a replication origin formed by four 22 bp tandem repeats. In silico analysis has identified plasmid encoded proteins to have a function in replication.

### DESCRIPTION OF THE INVENTION

Current invention discloses a method and its compounds for biological sample pretreatment that allows efficient release of the genomic DNA from cellular material. Major advantage of the described sample pretreatment method is its compatibility with downstream nucleic acid amplification procedures allowing detection of the target DNA from crude sample lysates. Thus current invention allows skipping of the DNA purification step prior to NAAT analysis, having therefore an important impact on the complexity and speed of the diagnostic technique.

The invention discloses cell lytic compounds that allow fast (within 5 min at RT° C.) and efficient release of the genomic material from mammalian cells, their pathogen and commensal microorganisms, bacterial and fungi cultures etc. Sample pretreatment buffer consists of membrane active (cell-penetrating) peptides, mild detergents or a combination of the above two.

Membrane active peptides have antibacterial and antimicrobial effect acting disruptively on bacterial membranes. They are also known as cell membrane penetrating agents that can deliver different cargo molecules into mammalian cells (eg oligonucleotides, siRNA, plasmids, peptides). Current invention targets novel usage of the cell-penetrating peptides for diagnostics purposes. At higher (μM-mM) concentrations cell-penetrating peptides disrupt cellular membranes, that allows the release of the genomic DNA that can be used as a target in the following nucleic acid amplification reaction. Cell membrane disruptive peptides have shown no or minimal inhibiting effect on nucleic acid amplification even at high concentrations, thus can be efficiently used as agents facilitating genomic material release.

Detergents are very good solubilizing agents, but they tend to denature proteins by destroying native three dimensional structures. Certain combination of the mild ionic or non-ionic detergents (eg Triton X-100, Triton X-114, NP-40, CHAPS, Octyl-β-glucoside, Octyl-β-thioglucopyronoside) at low (eg 0.1-1%) concentration allow efficient cell wall disruption in order to release genomic material enclosed within cells. These mild detergents do not interfere significantly with nucleic acid amplification procedure, and are able to induce or facilitate the release of the sufficient amount of the target nucleic acid. The composition and concentration of the detergents is set to efficiently lyse cells within 5 min RT° C. incubation.

The ability of the membrane active peptide and/or detergent mediated sample pretreatment to convert biological sample into material well usable for the nucleic acid amplification is the major focus of the invention and has been confirmed by establishing detection of the Chlamydia trachomatis, Mycoplasma genitalium and Homo sapiens genomic DNA from crude human urine lysates.

For that a diagnostic method for highly specific and sensitive C. trachomatis and M. genitalium detection from human samples has been developed based on isothermal nucleic acid amplification (RPA, LAMP) and including immunochromotographic product detection using lateral-flow strips. For both pathogens we have used double target system, where simultaneous detection of two different genomic targets is performed. This reduces probability of the false negative diagnostics test result in case deletions or mutations are introduced into pathogen genomic DNA regions used as the amplification targets. All target regions were selected based on their high homology among different pathogen strains and lack of identity with similar species.

For C. trachomatis detection we have used genomic sequence regions from a well-established diagnostic target—coding sequence 2 of the multicopy cryptic plasmid (CDS2). For the second target we have chosen β subunit of the tryptophan synthase gene TRPB. For M. genitalium detection we have used genomic sequence regions from gene encoding MgPa dominant adhesin (MGPB) that is the main component of multiple repeats throughout its genome. For the second target we used 16S rRNA gene that is also present in multiple copies within M. genitalium genome. M. genitalium 16S rRNA gene however is highly conserved between different Mycoplasma species (eg 98% identity with M. pneumoniae, 91% with M. gallisepticum). Thus multiple mutations containing regions were chosen for the isothermal amplification and additional specificity testing was performed for this particular target.

For each target, optimal primer pair combinations were established that enable highest sensitivity levels for the assay. Optimized RPA reaction allowed well detectable and stable product amplification with minimum of 20-50 target sequence copies. Optimized LAMP reaction with loop primers allowed product amplification with minimum of 5-10 target sequence copies. Each diagnostics target was tested for specificity of the reaction with 50 000 copies (0.16 ng) of H. sapiens genomic DNA and in case of M. genitalium 16S rRNA target also with 100 000 copies of M. pneumoniae genomic DNA. Isothermal amplification sensitivity and specificity was verified with total DNA extracted from human urine samples.

Major objective of the current invention was to develop a diagnostic assay applicable under point-of-care conditions. Thus we have integrated immunochromotographic amplification product detection into the diagnostics system. For that purpose, forward primer sequences were 5′ labeled with biotin and reverse primers with fluorescein amidite (FAM). During amplification reaction a dually labeled products were produced, that were detected within minutes using lateral-flow strips. Integration of the immunochromotographic product detection required additional primer optimization. Primers gaining template independent lateral-flow strip detectable signal were eliminated from the selection.

RPA and LAMP isothermal amplification based diagnostics methods were also showed to be suitable for simultaneous multiple target detection. Both assays were optimized for H. sapiens GAPDH gene target to be used as a positive control of the diagnostics test with human samples. PCR and isothermal amplification (RPA/LAMP/HDA) protocols were adjusted for optimal sensitivity and high specificity of the diagnostics test.

The present method for detection of nucleic acid target(s) from biological crude samples and body fluids comprises following steps:
a) sample pretreatment comprising cell lysis and release of nucleic acid targets in biological samples and body fluids such as tissue, urine, saliva, blood, stool, hair, etc. and their derivatives, but not limited to the examples list, wherein the lytic peptides are used to release nucleic acid targets in biological samples;
b) amplification of nucleic acid(s) comprising nucleic acid, such as DNA, RNA and their derivatives but not limited to the list, amplification initiated by presence of target and comprise amplification methods such as PCR (Polymerase Chain Reaction), HCR (Hybridization Chain Reaction), RCA (Rolling Circle Amplification), RPA (Recombinase Polymerase Amplification), LAMP (Loop mediated isothermal AMPlification), HDA (Helicase Dependent Amplification), etc. and their derivatives, but not limited to the examples list, wherein one or more specific target based sequences are amplified or sample solution obtained during the step (1) is directly subjected for further amplification procedure;
c) detection of amplification product(s) comprising the use of qualitative or quantitative detection methods such as sandwich assays, ELISAs (Enzyme Linked ImmunoSorbent Assay), LF (Lateral Flow) immunochromatographic assays, wavelength changing (visible spectrum, chemiluminescence, fluorescence, phosphorescence and etc.) dyes, denrimeres, etc. or corresponding moiety conjugated detector molecules and ligands, with or without optical apparatus, appropriate wavelength emitter or reader or their combination, wherein qualitative and quantitative detection is performed with crude sample solution.

The pretreatment method is specifically designed to detect nucleic acid target(s):
of Chlamydia trachomatis with the use of specific target region provided in Table 1 or with the use of specific primer(s) and/or its labeled derivative(s) sequences provided in Table 2, 3; and
Mycoplasma genitalium with the use of specific target region provided in Table 1 or with the use of specific primer(s) and/or its labeled derivative(s) sequences provided in Table 2, 3.

The present method with human genomic GAPDH target is used for detection:
as an internal validation and platform assessing technique;
as an internal validation and platform assessing technique with specific primer(s) and/or its labeled derivative(s) sequences provided in Tables 2, 3.

The pretreatment method that relates to molecular diagnostics of Chlamydia trachomatis wherein TRPB gene is used as molecular diagnostics target.

### EXAMPLES OF THE IMPLEMENTATION

### Example 1

### Fast Diagnostics of the Presence of Chlamydia trachomatis in a Urine Sample

Present protocol describes method and its components for highly sensitive Chlamydia trachomatis diagnostics from human urine sample. The whole procedure including sample pretreatment, target isothermal amplification and product detection takes under 20 min and requires 10 min incubation at 37° C. Described method detects two C. trachomatis targets TRPB sequence in the genomic region and CDS2 sequence in the cryptic plasmid region (Table 1).

TABLE 1
Genomic regions of Chlamydia trachomatis,
Mycoplasma genitalium and Homo sapiens used
for isothermal amplification based detection

| Target organism | Sequence name | Genebank accession nr |
|---|---|---|
| C. trachomatis | PL-CDS2 | FM865439.1 |
| | | sequence 756-1748 |
| | TRPB | FN652779.2 |
| | | sequence 193461-194639 |
| M. genitalium | 16S rRNA | CP003773.1 |
| | | sequence 169843-171366 |
| | MGPA | CP003773.1 |
| | | sequence 221365-225744 |
| H. sapiens | GAPDH | NG_007073.2 |

Both of the C. trachomatis targets are amplified using highly specific and sensitive primers that carry same labeling, forward primers are labeled with biotin and reverse with FAM. Thus C. trachomatis specific products are not distinguished during immunochromatographic detection on lateral-flow strips. Detection of the two C. trachomatis regions is used to ensure positive test results in case one of the target regions is mutated or deleted. The reaction also contains primers targeting H. sapiens GAPDH gene that produce DIG and FAM labeled product. This product is recognized as a separate lane on the lateral-flow strip and serves as a positive control for the whole procedure (release of the genomic material from cells, amplification and detection). Analytical sensitivity of the described method is 50 C. trachomatis cells and 50 H. sapiens cells per test. This allows detection of the C. trachomatis in the first void urine at pathogen concentration of 10 000 cells per 1 ml of urine or higher.

Patient urine sample is mixed with equal volume of sample pretreatment buffer containing 0.2% Triton X-114, 150 mM NaCl, 50 mM Tris pH 7.0, and incubated 5 min at RT° C. 10 μl of the treated sample is used in the RPA reaction containing following components: C. trachomatis PL-CDS2 5′ biotin labeled FW3 primer at 0.4 μM final concentration, C. trachomatis PL-CDS2 5′ FAM labeled RV1 primer at 0.4 μM final concentration, C. trachomatis TRPB 5′ biotin labeled FW2 primer at 0.4 μM final concentration, C. trachomatis TRPB 5′ FAM labeled RV3 primer at 0.4 μM final concentration, H. sapiens GAPDH 5′ DIG labeled FW3 primer at 0.4 μM final concentration, H. sapiens GAPDH 5′ FAM labeled RV2 primer at 0.4 μM final concentration (see Table 2 for primer sequences), 14 mM magnesium acetate, TwistDX RPA enzyme pellet and 29.5 μl of the rehydration buffer. Reaction is incubated at 37° C. for 10 min. The products are diluted 1:10 ratio with dilution buffer and analyzed on lateral-flow strips detecting Biotin-FAM and DIG-FAM labeled molecules.

TABLE 2
Specific primer sequences for recombinase polymerase amplification (RPA)
against targets provided in Table 1

| Target | | | SEQ |
|---|---|---|---|
| organism | | | ID |
| and region | | Sequence (5′-3′) | NO |
| C. trachomatis | Forward | FW1 5′- CTTCTTTGAAGCGTTGTCTTCTCGAGAAGATTT | 1 |
| PL-CDS2 | (FW) | FW2 5′- CTTCTCGAGAAGATTTATCGTACGCAAATATC | 2 |
| | primer | FW3 5′- | 3 |
| | sequences | CCTTCATTATGTCGGAGTCTGAGCACCCTAGGC | |
| | | FW4 5′- AGGCGTTTGTACTCCGTCACAGCGGTTGCTCG | 4 |
| | Reverse | RV1 5′- CTCTCAAGCAGGACTACAAGCTGCAATCCCTT | 5 |
| | (RV) | RV2 5′- ATGGTGGGGTTAAGGCAAATCGCCCGCACGTT | 6 |
| | primer | RV3 5′- TCT TCG TAA CTC GCT CCG GAA AAA TGG | 7 |
| | sequences | TGG GG | |
| | | RV4 5′- CTT TCT ACA AGA GTA CAT CGG TCA ACG AAG | 8 |
| | | AGG | |
| C. trachomatis | Forward | FW1 5′- ACT ATG CGG GGA GAC AAA CTC CTC TGA | 9 |
| TRPB | (FW) | CTG AAG | |
| | primer | FW2 5′- TCT TAA ACG CGA AGA TCT TTT GCA TAC AGG | 10 |
| | sequences | AGC | |
| | | FW3 5′- CAT ACA GGA GCA CAT AAA CTG AAT AAT GCT | 11 |
| | | CTT GG | |
| | | FW4 5′- CTC TTG GTC AGT GTT TGC TTG CTA AAT ATC | 12 |
| | | TTG | |
| | Reverse | RV1 5′- TCC CGC ACC TGT TTC AGC TAC AAC ACG TGT | 13 |
| | (RV) | TT | |
| | primer | RV2 5′- CTG TTG CTG TTG CTA CTC CAT GTT GTC CCG | 14 |
| | sequences | CAC | |
| | | RV3 5′- TCC CAT GTA TAC TAC ACA ATC TAA TCC TAG | 15 |
| | | ATA | |
| | | RV4 5′- TTC TGT CGT TCC ACA TCT TTT GCT CCC ATG | 16 |
| | | TAT | |
| M. genitalium | Forward | FW1 5′- AGC GCA ACC CTT ATC GTT AGT TAC ATT GTT | 17 |
| 16S rRNA | (FW) | TAA | |
| | primer | FW2 5′- CGT TAG TTA CAT TGT TTA ACG AGA CTG CTA | 18 |
| | sequences | ATG T | |
| | | FW3 5′- ACG TGC TAC AAT GGC CAA TAC AAA CAG | 19 |
| | | TAG CCA A | |
| | Reverse | RV1 5′- TTG CAG CCC TCA ATC CGA ACT GAG ACC | 20 |
| | (RV) | AAC TTT T | |
| | primer | RV2 5′- CAT AGC TGA TTC GCG ATT ACT AGT GAT TCC | 21 |
| | sequences | AGC | |
| | | RV3 5′- TTC CAA TAA AGG TTA GCA ACA CGT TTT TAA | 22 |
| | | ATA | |
| M. genitalium | Forward | FW1 5′- TTGGACTTGAAACAATAACAACTTCTCTTCACT | 23 |
| MGPA | (FW) | FW2 5′- | 24 |
| | primer | AAGATTACTGGAGAGAACCCAGGATCATTTGGA | |
| | sequences | FW3 5′- CAG TGG GCA GAC TAT GTC TTA CCT TTG ATT | 25 |
| | | GTA | |
| | | FW4 5′- TTA TCC TTA GTG TTA CTT TGG GAT TAA CGA | 26 |
| | | TTG G | |
| | | FW5 5′- | 27 |
| | | CAATGCACAGAAACAAAAAGGCATTACAAGCAGGG | |
| | Reverse | RV1 5′- TCT GAT TGC AAA GTT TTG CTG ACC ATC AAG | 28 |
| | (RV) | GTA | |
| | primer | RV2 5′- CTC TAC CGT TGT TAT CAT ACC TTC TGA TTG | 29 |
| | sequences | C | |
| | | RV3 5′- TTC TGT TAA TGA TCT CTT TAA AGA CAC TAC | 30 |
| | | CAA | |
| | | RV4 5′- CTT AGG AGC GTT AGA GAT CCC TGT TCT GTT | 31 |
| | | AAT G | |
| | | RV5 5′- CTT GTT TTA ACT TCT TAG GAG CGT TAG AGA | 32 |
| | | TCC C | |
| | | RV6 5′- | 33 |
| | | TTACTGGAGGTTTTGGTGGGGTTTTAGGAGTTGG | |
| H. sapiens | Forward | FW1 5′- | 34 |
| GAPDH | (FW) | CTCCTCCGGGTGATGCTTTTCCTAGATTATTCTC | |
| | primer | FW2 5′- CTA ACC CTG CGC TCC TGC CTC GAT GGG | 35 |
| | sequences | TGG AG | |
| | | FW3 5′- AAG TCA GGT GGA GCG AGG CTA GCT GGC | 36 |
| | | CCG ATT | |
| | Reverse | RV1 5′- TCC TTT TCC AAC TAC CCA TGA CTC AGC TTC | 37 |
| | (RV) | TCC C | |
| | primer | RV2 5′- CAC CAT GCC ACA GCC ACC ACA CCT CTG | 38 |
| | sequences | CGG GGA | |
| | | RV3 5′- CCA CCA CCA GAG GGG CCA TTT TGC GGT | 39 |
| | | GGA AAT | |

Chlamydia tests positive if the test gives 2 lines (Biotin-FAM and DIG-FAM), negative if the test gives 1 line DIG-FAM. The results of the test are invalid if none of the lines are present or only Biotin-FAM line is present.

### Example 2

### Fast Diagnostics of the Presence of Mycoplasma genitalium in a Urine Sample

Present protocol describes method and its components for highly sensitive Mycoplasma genitalium diagnostics from human urine sample. The whole procedure including sample pretreatment, target isothermal amplification and product detection takes under 20 min and requires 10 min incubation at 37° C. Described method detects two M. genitalium targets MGPA and 16S rRNA sequences in the pathogen genome (Table 1). Both of the M. genitalium targets are amplified using highly specific and sensitive primers that carry same labeling, forward primers are labeled with biotin and reverse with FAM. Thus M. genitalium specific products are not distinguished during immunochromatographic detection on lateral-flow strips.

Detection of the two M. genitalium regions is used to ensure positive test results in case one of the target regions is mutated or deleted. The reaction also contains primers targeting H. sapiens GAPDH gene that produce DIG and FAM labeled product. This product is recognized as a separate lane on the lateral-flow strip and serves as a positive control for the whole procedure (release of the genomic material from cells, amplification and detection). Analytical sensitivity of the described method is at least 50 M. genitalium cells and 50 H. sapiens cells per test. This allows detection of the M. genitalium in the first void urine at pathogen concentration of 10 000 cells per 1 ml of urine or higher.

Patient urine sample is mixed with equal volume of sample pretreatment buffer containing 0.2% NP-40, 150 mM NaCl, 50 mM Tris pH 7.0, and incubated 5 min at RT° C. 10 μl of the treated sample is used in the RPA reaction containing following components: M. genitalium MGPA 5′ biotin labeled FW4 primer at 0.4 μM final concentration, M. genitalium MGPA 5′ FAM labeled RV4 primer at 0.4 μM final concentration, M. genitalium 16S rRNA 5′ biotin labeled FW1 primer at 0.4 μM final concentration, M. genitalium 16S rRNA 5′ FAM labeled RV1 primer at 0.4 μM final concentration, H. sapiens GAPDH 5′ DIG labeled FW3 primer at 0.4 μM final concentration, H. sapiens GAPDH 5′ FAM labeled RV2 primer at 0.4 μM final concentration (see Table 2 for primer sequences), 14 mM magnesium acetate, TwistDX RPA enzyme pellet and 29.5 μl of the rehydration buffer. Reaction is incubated at 37° C. for 10 min. The products are diluted 1:10 ratio with dilution buffer and analyzed on lateral-flow strips detecting Biotin-FAM and DIG-FAM labeled molecules.

M. genitalium tests positive if the test gives 2 lines (Biotin-FAM and DIG-FAM), negative if the test gives 1 line DIG-FAM. The results of the test are invalid if none of the lines are present or only Biotin-FAM line is present

### Example 3

### Highly Sensitive Diagnostics of the Presence of Chlamydia trachomatis from a Patient Sample Extracted Total DNA

Present method uses highly sensitive loop mediated isothermal amplification (LAMP) for specific detection of C. trachomatis DNA. Analytical sensitivity of the described method is at least 5 C. trachomatis cells per test. LAMP reaction is prepared as follows: C. trachomatis PL-CDS2 SET4 primers F3 and B3 at 0.2 μM concentration each, C. trachomatis PL-CDS2 SET4 5′ biotin labeled FIP and 5′ FAM labeled BIP primers at 1.6 μM each, C. trachomatis PL-CDS2 SET4 5′ biotin labeled LF and 5′ FAM labeled LB loop primers at 0.8 μM each (see Table 3 for primer sequences), 5.6 μM dNTP, 6 mM MgSO₄, 0.8 M betain, 8 units of Bst polymerase, 2.5 μl of 10× Bst polymerase buffer and 5 μl of total DNA extracted from patient sample per 25 μl reaction. Incubate reaction for 1 h at 63° C., dilute diluted 1:10 ratio with dilution buffer and analyzed on lateral-flow strips detecting Biotin-FAM labeled molecules.

In a parallel reaction C. trachomatis TRPB targeting LAMP can be performed with SET1 primers (Table 3) for additional positive control (with analytical sensitivity of at least 5 C. trachomatis cells per test). Additionally H. sapiens GAPDH targeting LAMP with SET 1 primers (Table 3) could be used as a positive control of the reaction.

TABLE 3
Specific primer sequences for loop mediated isothermal amplification
(LAMP) against targets provided in Table 1

| Target | | | | SEQ |
|---|---|---|---|---|
| organism and | | | | ID |
| region | | | Sequence (5′-3′) | NO: |
| C. trachomatis | SET | F3 | GCTTGTTGGAAACAAATCTGA | 40 |
| PL-CDS2 | 1 | B3 | TCGAACATTTTTTAAAACCAGG | 41 |
| | | FIP | GATCGCCCAGACAATGCTCCTAATCTCCAAGCTTAAGACTTCA | 42 |
| | | BIP | AACCAATCCCGGGCATTGATAAAAACGGATGCGATGAAC | 43 |
| | SET | F3 | AAAGTGCATAAACTTCTGAGG | 44 |
| | 2 | B3 | CTAAAAAAAATCAATGCCCGG | 45 |
| | | FIP | TGTTTCCAACAAGCTACCATTTCTTATAATCCTCTTTTCTGTCTGACG | 46 |
| | | BIP | AATCTCCAAGCTTAAGACTTCAGAGATTGGTTGATCGCCCAGA | 47 |
| | SET | F3 | TCTAAAGACAAAAAAGATCCTCG | 48 |
| | 3 | B3 | TGTGATGGGTAAAGGGATT | 49 |
| | | FIP | GCATGAAAAGCTTCTCCTTATTCGAATGATCTACAAGTATGTTTGTTGAG | 50 |
| | | BIP | CCAATAGGATTCTTGGCGAATTTTTTGCAGCAAGAAATGTCGTTA | 51 |
| | SET | F3 | CGACTATTTTCTTGTTTAGAAGGTT | 52 |
| | 4 | B3 | GAAAAGATTGGTCTATTGTCCT | 53 |
| | | FIP | AGCAGCAAGCTATATTTCCTTAACAGCTATAGCGACTATTCCTTGA | 54 |
| | | BIP | GTCTTGGCAGAGGAAACTTTTTTAATGGATATGAATCTGCAAGAGTT | 55 |
| | | LF1 | GATTCCTAAACAGGATGAC | 56 |
| | | LB1 | TCGCATCTAGGATTAGAT | 57 |
| | | LF3 | AGATTCCTAAACAGGATGAC | 58 |
| | | LB2 | CGCATCTAGGATTAGATTATG | 59 |
| | SET | F3 | AATATCATCTTTGCGGTTGC | 60 |
| | 5 | B3 | TCTACAAGAGTACATCGGTCA | 61 |
| | | FIP | TCGAGCAACCGCTGTGACGACCTTCATTATGTCGGAGTC | 62 |
| | | BIP | GCAGCTTGTAGTCCTGCTTGAGTCTTCGTAACTCGCTCC | 63 |
| | | LF | TAC AAA CGC CTA GGG TGC | 64 |
| | | LB | CGG GCG ATT TGC CTT AAC | 65 |
| C. trachomatis | SET | F3 | GCA GTT GCA GGA AGA GAT C | 66 |
| TRPB | 1 | B3 | GTC ATC TTG AAG AAG ATA CGA A | 67 |
| | | FIP | GGA CTT TTG GAT TCG GGA TAA AAT GCT GAT | 68 |
| | | | ATT CTG ATT GCA TGT ATC G | |
| | | BIP | GGA GGA CTG GGC ATT TCT TCA TGG AAT ACT | 69 |
| | | | CCA GGT CGC | |
| | | LF1 | AGCGTTGGAGCCACCTC | 70 |
| | | LB1 | GAAAACATGCAGCACGTTTTGCA | 71 |
| | | LF2 | CAATAGCGTTGGAGCCACCT | 72 |
| | | LB2 | AACATGCAGCACGTTTTGCA | 73 |
| | SET | F3 | CAAGATGACGATGGACAAGT | 74 |
| | 2 | B3 | CCAGATAAGTTAACGATGACGA | 75 |
| | | FIP | GGCTCGTCCTGACTCATGCTCCGCTGGATTAGATTATCCT | 76 |
| | | BIP | CCGATGAAGAGGCGTTACGAGGAGCATGTGAAGA CTCCAAT | 77 |
| | | LF | CAT GAT CTG GCC CAA CTG A | 78 |
| | | LB | TCC TGC TTA CTA GAA ATG AGG G | 79 |
| M. genitalium | SET | F3 | ATTGGTTAACTTACCTAGTGGC | 80 |
| MGPA | 1 | B3 | ACTTCTTAGGAGCGTTAGAGA | 81 |
| | | FIP | GACATAGTCTGCCCACTGGTTGATCCTCAAACCCAACAGTT | 82 |
| | | BIP | AGGCATTACAAGCAGGGTTTGAAAGACACTACCAACTGCTT | 83 |
| | | LF | AAAGGGTTGAAAGACAGTTTGG | 84 |
| | | LB | AAGGTTGATGTCTTGACCA | 85 |
| | | F3 | CACCTTACCAGTAACTGAACT | 86 |
| | SET | B3 | AACCCTGCTTGTAATGCC | 87 |
| | 2 | FIP | TTAAGCGGATTGAAGCTTGATCTGTCTATGACCAGTATGTACCA | 88 |
| | | BIP | CCCAACAGTTTATCCCGGTACTAAGGTAAGACATAGTCTGCC | 89 |
| | | LF | GCCACTAGGTAAGTTAACCAAT | 90 |
| | | LB | AATGCATCAAGTACAGGTCC | 91 |
| | SET | F3 | CACCTTACCAGTAACTGAACT | 92 |
| | 3 | B3 | AACCCTGCTTGTAATGCC | 93 |
| | | FIP | TTAAGCGGATTGAAGCTTGATCTCTATGACCAGTATGTACCACT | 94 |
| | | BIP | CCCAACAGTTTATCCCGGTACTAAGGTAAGACATAGTCTGCC | 95 |
| | | LF | GCCACTAGGTAAGTTAACCAAT | 96 |
| | | LB | AATGCATCAAGTACAGGTCC | 97 |
| | SET | F3 | CACCTTACCAGTAACTGAACT | 98 |
| | 4 | B3 | AACCCTGCTTGTAATGCC | 99 |
| | | FIP | TTAAGCGGATTGAAGCTTGATCGTCTATGACCAGTATGTACCAC | 100 |
| | | BIP | CCCAACAGTTTATCCCGGTACTAAGGTAAGACATAGTCTGCC | 101 |
| | | LF | GCCACTAGGTAAGTTAACCAAT | 102 |
| | | LB | AATGCATCAAGTACAGGTCC | 203 |
| | SET | F3 | GATCCTCAAACCCAACAGTT | 104 |
| | 5 | B3 | TTAGGAGTTGGTTTGGTTGG | 105 |
| | | FIP | GACATAGTCTGCCCACTGGTTTGCATCAAGTACAGGTCC | 106 |
| | | BIP | AGGCATTACAAGCAGGGTTTGAACTTCTTAGGAGCGTTAGAGA | 107 |
| | | LF | AAAGGGTTGAAAGACAGTTTGG | 108 |
| | | LB | AAGGTTGATGTCTTGACCAA | 109 |
| | SET | F3 | TGTCTATGACCAGTATGTACCA | 110 |
| | 6 | B3 | AACCCTGCTTGTAATGCC | 111 |
| | | FIP | ACTGTTGGGTTTGAGGATCTTTATTGGTTAACTTACCTAGTGGC | 112 |
| | | BIP | CCCGGTACTAAATGCATCAAGTAAGGTAAGACATAGTCTGCC | 113 |
| | | LF | TTAAGCGGATTGAAGCTTGATC | 114 |
| | | LB | CCAAACTGTCTTTCAACCCTTT | 115 |
| | SET | F3 | CACCTTACCAGTAACTGAACT | 116 |
| | 7 | B3 | AACCCTGCTTGTAATGCC | 117 |
| | | FIP | TTACCTTTAAGCGGATTGAAGCTGACCAGTATGTACCACTATTG | 118 |
| | | BIP | CCCAACAGTTTATCCCGGTACTAAGGTAAGACATAGTCTGCC | 119 |
| | | LF | GATCAAAGCCACTAGGTAAGTT | 120 |
| | | LB | AATGCATCAAGTACAGGTCC | 121 |
| | SET | F3 | CTATGACCAGTATGTACCACTA | 122 |
| | 8 | B3 | AACCCTGCTTGTAATGCC | 123 |
| | | FIP | ACTGTTGGGTTTGAGGATCTTTTTGGTTAACTTACCTAGTGGC | 124 |
| | | BIP | CCCGGTACTAAATGCATCAAGTAAGGTAAGACATAGTCTGCC | 125 |
| | | LF | TTAAGCGGATTGAAGCTTGATC | 126 |
| | | LB | CCAAACTGTCTTTCAACCCTTT | 127 |
| | SET | F3 | TGACCAGTATGTACCACTAT | 128 |
| | 9 | B3 | AACCCTGCTTGTAATGCC | 129 |
| | | FIP | ACTGTTGGGTTTGAGGATCTTTTGGTTAACTTACCTAGTGGCT | 130 |
| | | BIP | CCCGGTACTAAATGCATCAAGTAAGGTAAGACATAGTCTGCC | 131 |
| | | LF | TTAAGCGGATTGAAGCTTGATC | 132 |
| | | LB | CCAAACTGTCTTTCAACCCTTT | 133 |
| | SET | F3 | TGACCAGTATGTACCACTATTG | 134 |
| | 10 | B3 | AACCCTGCTTGTAATGCC | 135 |
| | | FIP | ACTGTTGGGTTTGAGGATCTTTGTTAACTTACCTAGTGGCTT | 136 |
| | | BIP | CCCGGTACTAAATGCATCAAGTAAGGTAAGACATAGTCTGCC | 137 |
| | | LF | TTAAGCGGATTGAAGCTTGATC | 138 |
| | | LB | CCAAACTGTCTTTCAACCCTTT | 139 |
| | SET | F3 | GACCAGTATGTACCACTATT | 140 |
| | 11 | B3 | AACCCTGCTTGTAATGCC | 141 |
| | | FIP | ACTGTTGGGTTTGAGGATCTTTGGTTAACTTACCTAGTGGCTT | 142 |
| | | BIP | CCCGGTACTAAATGCATCAAGTAAGGTAAGACATAGTCTGCC | 143 |
| | | LF | TTAAGCGGATTGAAGCTTGATC | 144 |
| | | LB | CCAAACTGTCTTTCAACCCTTT | 145 |
| M. genitalium | SET | F3 | CGTGAACGATGAAGGTCTT | 146 |
| 16S rRNA | 1 | B3 | ACCACACTCTAGACTGATAGTT | 147 |
| | | FIP | GCGACTGCTGGCACATAGTTAAGAATGACTCTAGCAGGCA | 148 |
| | | BIP | ACATAGGTCGCAAGCGTTATCCCTGCCTTTAACACCAGACTT | 149 |
| | | LF | GTACAGTCAAACTCCAGCCA | 150 |
| | | LB | GGATTTATTGGGCGTAAAGCAA | 151 |
| | SET | F3 | CGTGAACGATGAAGGTCTT | 152 |
| | 2 | B3 | ACCACACTCTAGACTGATAGTT | 153 |
| | | FIP | GCGACTGCTGGCACATAGTAATGACTCTAGCAGGCAATG | 154 |
| | | BIP | ACATAGGTCGCAAGCGTTATCCCTGCCTTTAACACCAGACTT | 155 |
| | | LF | TGGTACAGTCAAACTCCAGC | 156 |
| | | LB | GGATTTATTGGGCGTAAAGCAA | 157 |
| | SET | F3 | CGTGAACGATGAAGGTCTT | 158 |
| | 3 | B3 | ACCACACTCTAGACTGATAGTT | 159 |
| | | FIP | GCTGGCACATAGTTAGTCGTCAGAAGAATGACTCTAGCAGGC | 160 |
| | | BIP | ACATAGGTCGCAAGCGTTATCCCTGCCTTTAACACCAGACTT | 161 |
| | | LF | GTACAGTCAAACTCCAGCCA | 162 |
| | | LB | GGATTTATTGGGCGTAAAGCAA | 163 |
| | SET | F3 | CGTGAACGATGAAGGTCTT | 164 |
| | 4 | B3 | ACCACACTCTAGACTGATAGTT | 165 |
| | | FIP | GCGACTGCTGGCACATAGTTAGAATGACTCTAGCAGGCAAT | 166 |
| | | BIP | ACATAGGTCGCAAGCGTTATCCCTGCCTTTAACACCAGACTT | 167 |
| | | LF | TGGTACAGTCAAACTCCAGC | 168 |
| | | LB | GGATTTATTGGGCGTAAAGCAA | 169 |
| | SET | F3 | CATTACTGACGCTTAGGCTT | 170 |
| | 5 | B3 | GCCAAGGATGTCAAGTCTAG | 171 |
| | | FIP | CTTCACTACCGAAGGGATCGCCCTAGTAGTCCACACCGTAA | 172 |
| | | BIP | GCCTGGGTAGTACATTCGCAAAACATGCTCCACCACTTG | 173 |
| | | LF | TCCGACAGCTAGTATCTATCGT | 174 |
| | | LB | TGAAACTCAAACGGAATTGACG | 175 |
| | SET | F3 | CGTGAACGATGAAGGTCTT | 176 |
| | 6 | B3 | ACCACACTCTAGACTGATAGTT | 177 |
| | | FIP | GCGACTGCTGGCACATAGTGACTCTAGCAGGCAATGG | 178 |
| | | BIP | ACATAGGTCGCAAGCGTTATCCCTGCCTTTAACACCAGACTT | 179 |
| | | LF | AAAGTGGTACAGTCAAACTCCA | 180 |
| | | LB | GGATTTATTGGGCGTAAAGCAA | 181 |
| | SET | F3 | CAAGTGGTGGAGCATGTT | 182 |
| | 7 | B3 | TCCCTTCCTTCCTCCAATT | 183 |
| | | FIP | CGACAACCATGCACCACCTCTAGACTTGACATCCTTGGC | 184 |
| | | BIP | CAGCTCGTGTCGTGAGATGTTTAACTAACGATAAGGGTTGCG | 185 |
| | | LF | GTCACTCGGTTAACCTCCATT | 186 |
| | | LB | GGTTAAGTCCCGCAACGA | 187 |
| | SET | F3 | AATGACTCTAGCAGGCAATG | 188 |
| | 8 | B3 | ACCACACTCTAGACTGATAGTT | 189 |
| | | FIP | CGGATAACGCTTGCGACCTTAAGTGACGACTAACTATGTGC | 190 |
| | | BIP | AAGCGCAGGCGGATTGAACCAATGCATACAACTGTTAAGC | 191 |
| | | LF | TGTATTACCGCGACTGCTG | 192 |
| | | LB | AGTCTGGTGTTAAAGGCAGC | 193 |
| | SET | F3 | AATGACTCTAGCAGGCAATG | 194 |
| | 9 | B3 | ACCACACTCTAGACTGATAGTT | 195 |
| | | FIP | CGGATAACGCTTGCGACCTAAGTGACGACTAACTATGTGC | 196 |
| | | BIP | AAGCGCAGGCGGATTGAACCAATGCATACAACTGTTAAGC | 197 |
| | | LF | TGTATTACCGCGACTGCTG | 198 |
| | | LB | AGTCTGGTGTTAAAGGCAGC | 199 |
| | SET | F3 | CAAGTGGTGGAGCATGTT | 200 |
| | 10 | B3 | GTTTGCAGCCCTAGACATAA | 201 |
| | | FIP | CGACACGAGCTGACGACAACCTTGGCAAAGTTATGGAAAC | 202 |
| | | BIP | TGGGTTAAGTCCCGCAACGCCAATTTACATTAGCAGTCTCG | 203 |
| | | LF | CATGCACCACCTGTCACT | 204 |
| | | LB | CGCAACCCTTATCGTTAGTTAC | 205 |
| | SET | F3 | CGCATAAGAACTTTAGTTCGC | 206 |
| | 11 | B3 | AAGACCTTCATCGTTCACG | 207 |
| | | FIP | TAGCTACACGTCATTGCCTTGGAGGGTTCGTTATTTGATGAGG | 208 |
| | | BIP | CACAATGGGACTGAGACACGGAGCTTTCGCTCATTGTGAA | 209 |
| | | LF | CCTACCAACTAGCTGATATGGC | 210 |
| | | LB | TACTCCTACGGGAGGCAG | 211 |
| H. sapiens | SET | F3 | TGGGTGTGAACCATGAGA | 212 |
| GAPDH | 1 | B3 | AGTCCTTCCACGATACCAA | 213 |
| | | FIP | TCCATAGGGTGCCAGGCTGTATGACAACAGCCTCA AGAT | 214 |
| | | BIP | CTTTCTTTGCAGCAATGCCTCCAGTTGTCATGGATGACCTTG | 215 |
| | | LF | CTG CCT TCC TCA CCT GAT G | 216 |
| | | LB | TGC ACC ACC AAC TGC TTA | 217 |
| | SET | F3 | CCCCAAAGGCCAGGCT | 218 |
| | 2 | B3 | AGAAGGGATGGGAGAGAGC | 219 |
| | | FIP | GGAATGGGGAGAAGGGCAGGTTAAATGTCACCGGGAGGATTG | 220 |
| | | BIP | CGGAAACCAGATCTCCCACCGGCTACAGAAAGGTCAGCAGC | 221 |
| | SET | F3 | ATCAAGTGGGGCGATGCT | 222 |
| | 3 | B3 | GGGCAGAGATGATGACCCT | 223 |
| | | FIP | GCACTCACCCCAGCCTTCTCGCTGAGTACGTCGTGGAGT | 224 |
| | | BIP | AAGCTGACTCAGCCCTGCAAACCCTGCAAATGAGCCTACA | 225 |
| | | F3 | GTT GAC CCG ACC CCA AAG | 226 |
| | | B3 | AAG GGA TGG GAG AGA GCC | 227 |
| | | FIP | CGG AAT GGG GAG AAG GGC AGA TGT CAC CGG | 228 |
| | | | GAG GAT TGG | |
| | | BIP | CGG AAA CCA GAT CTC CCA CCG CCA GCT ACA | 229 |
| | | | GAA AGG TCA GC | |

1. A method for detection of nucleic acid targets from biological samples and body fluids comprising the steps of:
a) sample pretreatment comprising cell lysis and release of nucleic acid targets in biological samples and body fluids;
b) amplification of nucleic acid(s);
c) detection of amplification product(s),
wherein lytic peptides are used to release nucleic acid targets in biological samples or body fluids.

2. The method according to claim 1, wherein detergents are used to release nucleic acid targets in biological samples or body fluids.

3. The method according to claim 1, wherein combination of lytic peptides and detergents are used to release nucleic acid targets in biological samples or body fluids.

4. The method according to claim 1, wherein one or more specific target based sequences are amplified.

5. The method according to claim 1, wherein sample solution obtained during the step a) is directly subjected for further amplification procedure.

6. The method according to claim 1, wherein qualitative and quantitative detection is performed with crude sample solution.

7. The method according to claim 1, wherein the Chlamydia trachomatis nucleic acid target(s) with the use of specific target region provided in Table 1 is detected.

8. The method according to claim 1, wherein the Mycoplasma genitalium nucleic acid target(s) with the use of specific target region provided in Table 1 is detected.

9. The method according to claim 1, wherein the Chlamydia trachomatis nucleic acid target(s) with the use of specific primer(s) and/or its labeled derivative(s) sequences provided in Table 2 and 3 is detected.

10. The method according to claim 1, wherein the Mycoplasma genitalium nucleic acid target(s) with the use of specific primer(s) and/or its labeled derivative(s) sequences provided in Table 2 and 3 is detected.

11. The method according to claim 1, wherein the human genomic GAPDH target is used for detection as an internal validation and platform assessing technique.

12. The method according to claim 1, wherein the human genomic GAPDH target is used for detection as an internal validation and platform assessing technique with specific primer(s) and/or its labeled derivative(s) sequences provided in Tables 2, 3.

13. A molecular diagnostics method of Chlamydia trachomatis, wherein the TRPB gene is used as molecular diagnostics target.


SEQUENCE LISTING (229 sequences)

SEQ ID NO: 1 - DNA, 33 bp, artificial sequence
  chemically synthesized
cttctttgaagcgttgtcttctcgagaagattt

SEQ ID NO: 2 - DNA, 32 bp, artificial sequence
  chemically synthesized
cttctcgagaagatttatcgtacgcaaatatc

SEQ ID NO: 3 - DNA, 33 bp, artificial sequence
  chemically synthesized
ccttcattatgtcggagtctgagcaccctaggc

SEQ ID NO: 4 - DNA, 32 bp, artificial sequence
  chemically synthesized
aggcgtttgtactccgtcacagcggttgctcg

SEQ ID NO: 5 - DNA, 32 bp, artificial sequence
  chemically synthesized
ctctcaagcaggactacaagctgcaatccctt

SEQ ID NO: 6 - DNA, 32 bp, artificial sequence
  chemically synthesized
atggtggggttaaggcaaatcgcccgcacgtt

SEQ ID NO: 7 - DNA, 32 bp, artificial sequence
  chemically synthesized
tcttcgtaactcgctccggaaaaatggtgggg

SEQ ID NO: 8 - DNA, 33 bp, artificial sequence
  chemically synthesized
ctttctacaagagtacatcggtcaacgaagagg

SEQ ID NO: 9 - DNA, 33 bp, artificial sequence
  chemically synthesized
actatgcggggagacaaactcctctgactgaag

SEQ ID NO: 10 - DNA, 33 bp, artificial sequence
  chemically synthesized
tcttaaacgcgaagatcttttgcatacaggagc

SEQ ID NO: 11 - DNA, 35 bp, artificial sequence
  chemically synthesized
catacaggagcacataaactgaataatgctcttgg

SEQ ID NO: 12 - DNA, 33 bp, artificial sequence
  chemcially synthesized
ctcttggtcagtgtttgcttgctaaatatcttg

SEQ ID NO: 13 - DNA, 32 bp, artificial sequence
  chemically synthesized
tcccgcacctgtttcagctacaacacgtgttt

SEQ ID NO: 14 - DNA, 33 bp, artificial sequence
  chemically synthesized
ctgttgctgttgctactccatgttgtcccgcac

SEQ ID NO: 15 - DNA, 33 bp, artificial sequence
  chemically synthesized
tcccatgtatactacacaatctaatcctagata

SEQ ID NO: 16 - DNA, 33 bp, artificial sequence
  chemically synthesized
ttctgtcgttccacatcttttgctcccatgtat

SEQ ID NO: 17 - DNA, 33 bp, artificial sequence
  chemically synthesized
agcgcaacccttatcgttagttacattgtttaa

SEQ ID NO: 18 - DNA, 34 bp, artificial sequence
  chemically synthesized
cgttagttacattgtttaacgagactgctaatgt

SEQ ID NO: 19 - DNA, 34 bp, artificial sequence
  chemically synthesized
acgtgctacaatggccaatacaaacagtagccaa

SEQ ID NO: 20 - DNA, 34 bp, artificial sequence
  chemically synthesized
ttgcagccctcaatccgaactgagaccaactttt

SEQ ID NO: 21 - DNA, 33 bp, artificial sequence
  chemcially synthesized
catagctgattcgcgattactagtgattccagc

SEQ ID NO: 22 - DNA, 33 bp, artificial sequence
  chemically synthesized
ttccaataaaggttagcaacacgtttttaaata

SEQ ID NO: 23 - DNA, 33 bp, artificial sequence
  chemically synthesized
ttggacttgaaacaataacaacttctcttcact

SEQ ID NO: 24 - DNA, 33 bp, artificial sequence
  chemically synthesized
aagattactggagagaacccaggatcatttgga

SEQ ID NO: 25 - DNA, 33 bp, artificial sequence
  chemically synthesized
cagtgggcagactatgtcttacctttgattgta

SEQ ID NO: 26 - DNA, 34 bp, artificial sequence
  chemically synthesized
ttatccttagtgttactttgggattaacgattgg

SEQ ID NO: 27 - DNA, 35 bp, artificial sequence
  chemically synthesized
caatgcacagaaacaaaaaggcattacaagcaggg

SEQ ID NO: 28 - DNA, 33 bp, artificial sequence
  chemically synthesized
tctgattgcaaagttttgctgaccatcaaggta

SEQ ID NO: 29 - DNA, 31 bp, artificial sequence
  chemically synthesized
ctctaccgttgttatcataccttctgattgc

SEQ ID NO: 30 - DNA, 33 bp, artificial sequence
  chemically synthesized
ttctgttaatgatctctttaaagacactaccaa

SEQ ID NO: 31 - DNA, 34 bp, artificial sequence
  chemically synthesized
cttaggagcgttagagatccctgttctgttaatg

SEQ ID NO: 32 - DNA, 34 bp, artificial sequence
  chemcially synthesized
cttgttttaacttcttaggagcgttagagatccc

SEQ ID NO: 33 - DNA, 34 bp, artificial sequence
  chemically synthesized
ttactggaggttttggtggggttttaggagttgg

SEQ ID NO: 34 - DNA, 34 bp, artificial sequence
  chemically synthesized
ctcctccgggtgatgcttttcctagattattctc

SEQ ID NO: 35 - DNA, 32 bp, artificial sequence
  chemically synthesized
ctaaccctgcgctcctgcctcgatgggtggag

SEQ ID NO: 36 - DNA, 33 bp, artificial sequence
  chemically synthesized
aagtcaggtggagcgaggctagctggcccgatt

SEQ ID NO: 37 - DNA, 34 bp, artificial sequence
  chemically synthesized
tccttttccaactacccatgactcagcttctccc

SEQ ID NO: 38 - DNA, 33 bp, artificial sequence
  chemically synthesized
caccatgccacagccaccacacctctgcgggga

SEQ ID NO: 39 - DNA, 33 bp, artificial sequence
  chemically synthesized
ccaccaccagaggggccattttgcggtggaaat

SEQ ID NO: 40 - DNA, 21 bp, artificial sequence
  chemically synthesized
gcttgttggaaacaaatctga

SEQ ID NO: 41 - DNA, 22 bp, artificial sequence
  chemically synthesized
tcgaacattttttaaaaccagg

SEQ ID NO: 42 - DNA, 43 bp, artificial sequence
  chemically synthesized
gatcgcccagacaatgctcctaatctccaagcttaagacttca

SEQ ID NO: 43 - DNA, 39 bp, artificial sequence
  chemically synthesized
aaccaatcccgggcattgataaaaacggatgcgatgaac

SEQ ID NO: 44 - DNA, 21 bp, artificial sequence
  chemically synthesized
aaagtgcataaacttctgagg

SEQ ID NO: 45 - DNA, 21 bp, artificial sequence
  chemically synthesized
ctaaaaaaaatcaatgcccgg

SEQ ID NO: 46 - DNA, 48 bp, artificial sequence
  chemically synthesized
tgtttccaacaagctaccatttcttataatcctcttttctgtctgacg

SEQ ID NO: 47 - DNA, 43 bp, artificial sequence
  chemcially sytnhesized
aatctccaagcttaagacttcagagattggttgatcgcccaga

SEQ ID NO: 48 - DNA, 23 bp, artificial sequence
  chemically synthesized
tctaaagacaaaaaagatcctcg

SEQ ID NO: 49 - DNA, 19 bp, artificial sequence
  chemically synthesized
tgtgatgggtaaagggatt

SEQ ID NO: 50 - DNA, 50 bp, artificial sequence
  chemically synthesized
gcatgaaaagcttctccttattcgaatgatctacaagtatgtttgttgag

SEQ ID NO: 51 - DNA, 45 bp, artificial sequence
  chemically synthesized
ccaataggattcttggcgaattttttgcagcaagaaatgtcgtta

SEQ ID NO: 52 - DNA, 25 bp, artificial sequence
  chemically synthesized
cgactattttcttgtttagaaggtt

SEQ ID NO: 53 - DNA, 22 bp, artificial sequence
  chemically synthesized
gaaaagattggtctattgtcct

SEQ ID NO: 54 - DNA, 46 bp, artificial sequence
  chemically synthesized
agcagcaagctatatttccttaacagctatagcgactattccttga

SEQ ID NO: 55 - DNA, 47 bp, artificial sequence
  chemically synthesized
gtcttggcagaggaaacttttttaatggatatgaatctgcaagagtt

SEQ ID NO: 56 - DNA, 19 bp, artificial sequence
  chemically synthesized
gattcctaaacaggatgac

SEQ ID NO: 57 - DNA, 18 bp, artificial sequence
  chemically synthesized
tcgcatctaggattagat

SEQ ID NO: 58 - DNA, 20 bp, artificial sequence
  chemically synthesized
agattcctaaacaggatgac

SEQ ID NO: 59 - DNA, 21 bp, artificial sequence
  chemically synthesized
cgcatctaggattagattatg

SEQ ID NO: 60 - DNA, 20 bp, artificial sequence
  chemically synthesized
aatatcatctttgcggttgc

SEQ ID NO: 61 - DNA, 21 bp, artificial sequence
  chemically synthesized
tctacaagagtacatcggtca

SEQ ID NO: 62 - DNA, 39 bp, artificial sequence
  chemically synthesized
tcgagcaaccgctgtgacgaccttcattatgtcggagtc

SEQ ID NO: 63 - DNA, 39 bp, artificial sequence
  chemically synthesized
gcagcttgtagtcctgcttgagtcttcgtaactcgctcc

SEQ ID NO: 64 - DNA, 18 bp, artificial sequence
  chemcially synthesized
tacaaacgcctagggtgc

SEQ ID NO: 65 - DNA, 18 bp, artificial sequence
  chemically synthesized
cgggcgatttgccttaac

SEQ ID NO: 66 - DNA, 19 bp, artificial sequence
  chemically synthesized
gcagttgcaggaagagatc

SEQ ID NO: 67 - DNA, 22 bp, artificial sequence
  chemically synthesized
gtcatcttgaagaagatacgaa

SEQ ID NO: 68 - DNA, 49 bp, artificial sequence
  chemically synthesized
ggacttttggattcgggataaaatgctgatattctgattgcatgtatcg

SEQ ID NO: 69 - DNA, 39 bp, artificial sequence
  chemically synthesized
ggaggactgggcatttcttcatggaatactccaggtcgc

SEQ ID NO: 70 - DNA, 17 bp, artificial sequence
  chemically synthesized
agcgttggagccacctc

SEQ ID NO: 71 - DNA, 23 bp, artificial sequence
  chemically synthesized
gaaaacatgcagcacgttttgca

SEQ ID NO: 72 - DNA, 20 bp, artificial sequence
  chemically synthesized
caatagcgttggagccacct

SEQ ID NO: 73 - DNA, 20 bp, artificial sequence
  chemically synthesized
aacatgcagcacgttttgca

SEQ ID NO: 74 - DNA, 20 bp, artificial sequence
  chemically synthesized
caagatgacgatggacaagt

SEQ ID NO: 75 - DNA, 22 bp, artificial sequence
  chemically synthesized
ccagataagttaacgatgacga

SEQ ID NO: 76 - DNA, 40 bp, artificial sequence
  chemically synthesized
ggctcgtcctgactcatgctccgctggattagattatcct

SEQ ID NO: 77 - DNA, 41 bp, artificial sequence
  chemically synthesized
ccgatgaagaggcgttacgaggagcatgtgaagactccaat

SEQ ID NO: 78 - DNA, 19 bp, artificial sequence
  chemically synthesized
catgatctggcccaactga

SEQ ID NO: 79 - DNA, 22 bp, artificial sequence
  chemically synthesized
tcctgcttactagaaatgaggg

SEQ ID NO: 80 - DNA, 22 bp, artificial sequence
  chemically synthesized
attggttaacttacctagtggc

SEQ ID NO: 81 - DNA, 21 bp, artificial sequence
  chemically synthesized
acttcttaggagcgttagaga

SEQ ID NO: 82 - DNA, 41 bp, artificial sequence
  chemically synthesized
gacatagtctgcccactggttgatcctcaaacccaacagtt

SEQ ID NO: 83 - DNA, 41 bp, artificial sequence
  chemically synthesized
aggcattacaagcagggtttgaaagacactaccaactgctt

SEQ ID NO: 84 - DNA, 22 bp, artificial sequence
  chemically synthesized
aaagggttgaaagacagtttgg

SEQ ID NO: 85 - DNA, 19 bp, artificial sequence
  chemically synthesized
aaggttgatgtcttgacca

SEQ ID NO: 86 - DNA, 21 bp, artificial sequence
  chemically synthesized
caccttaccagtaactgaact

SEQ ID NO: 87 - DNA, 18 bp, artificial sequence
  chemically synthesized
aaccctgcttgtaatgcc

SEQ ID NO: 88 - DNA, 44 bp, artificial sequence
  chemically synthesized
ttaagcggattgaagcttgatctgtctatgaccagtatgtacca

SEQ ID NO: 89 - DNA, 42 bp, artificial sequence
  chemically synthesized
cccaacagtttatcccggtactaaggtaagacatagtctgcc

SEQ ID NO: 90 - DNA, 22 bp, artificial sequence
  chemically synthesized
gccactaggtaagttaaccaat

SEQ ID NO: 91 - DNA, 20 bp, artificial sequence
  chemically synthesized
aatgcatcaagtacaggtcc

SEQ ID NO: 92 - DNA, 21 bp, artificial sequence
  chemically synthesized
caccttaccagtaactgaact

SEQ ID NO: 93 - DNA, 18 bp, artificial sequence
  chemically synthesized
aaccctgcttgtaatgcc

SEQ ID NO: 94 - DNA, 44 bp, artificial sequence
  chemically synthezised
ttaagcggattgaagcttgatctctatgaccagtatgtaccact

SEQ ID NO: 95 - DNA, 42 bp, artificial sequence; identical to SEQ ID NO: 89
  chemically synthesized

SEQ ID NO: 96 - DNA, 22 bp, artificial sequence
  chemically synthesized
gccactaggtaagttaaccaat

SEQ ID NO: 97 - DNA, 20 bp, artificial sequence
  chemically synthesized
aatgcatcaagtacaggtcc

SEQ ID NO: 98 - DNA, 21 bp, artificial sequence
  chemically synthesized
caccttaccagtaactgaact

SEQ ID NO: 99 - DNA, 18 bp, artificial sequence
  chemically synthesized
aaccctgcttgtaatgcc

SEQ ID NO: 100 - DNA, 44 bp, artificial sequence
  chemically synthesized
ttaagcggattgaagcttgatcgtctatgaccagtatgtaccac

SEQ ID NO: 101 - DNA, 42 bp, artificial sequence; identical to SEQ ID NO: 89
  chemically synthesized

SEQ ID NO: 102 - DNA, 22 bp, artificial sequence
  chemically synthesized
gccactaggtaagttaaccaat

SEQ ID NO: 103 - DNA, 20 bp, artificial sequence
  chemically synthesized
aatgcatcaagtacaggtcc

SEQ ID NO: 104 - DNA, 20 bp, artificial sequence
  chemically synthesized
gatcctcaaacccaacagtt

SEQ ID NO: 105 - DNA, 20 bp, artificial sequence
  chemically synthesized
ttaggagttggtttggttgg

SEQ ID NO: 106 - DNA, 39 bp, artificial sequence
  chemically synthesized
gacatagtctgcccactggtttgcatcaagtacaggtcc

SEQ ID NO: 107 - DNA, 43 bp, artificial sequence
  chemically synthesized
aggcattacaagcagggtttgaacttcttaggagcgttagaga

SEQ ID NO: 108 - DNA, 22 bp, artificial sequence
  chemically synthesized
aaagggttgaaagacagtttgg

SEQ ID NO: 109 - DNA, 20 bp, artificial sequence
  chemically synthesized
aaggttgatgtcttgaccaa

SEQ ID NO: 110 - DNA, 22 bp, artificial sequence
  chemically synthesized
tgtctatgaccagtatgtacca

SEQ ID NO: 111 - DNA, 18 bp, artificial sequence
  chemically synthesized
aaccctgcttgtaatgcc

SEQ ID NO: 112 - DNA, 44 bp, artificial sequence
  chemically synthesized
actgttgggtttgaggatctttattggttaacttacctagtggc

SEQ ID NO: 113 - DNA, 42 bp, artificial sequence
  chemically synthesized
cccggtactaaatgcatcaagtaaggtaagacatagtctgcc

SEQ ID NO: 114 - DNA, 22 bp, artificial sequence
  chemically synthesized
ttaagcggattgaagcttgatc

SEQ ID NO: 115 - DNA, 22 bp, artificial sequence
  chemically synthesized
ccaaactgtctttcaacccttt

SEQ ID NO: 116 - DNA, 21 bp, artificial sequence
  chemically synthesized
caccttaccagtaactgaact

SEQ ID NO: 117 - DNA, 18 bp, artificial sequence
  chemically synthesized
aaccctgcttgtaatgcc

SEQ ID NO: 118 - DNA, 44 bp, artificial sequence
  chemically synthesized
ttacctttaagcggattgaagctgaccagtatgtaccactattg

SEQ ID NO: 119 - DNA, 42 bp, artificial sequence; identical to SEQ ID NO: 89
  chemically synthesized

SEQ ID NO: 120 - DNA, 22 bp, artificial sequence
  chemically synthesized
gatcaaagccactaggtaagtt

SEQ ID NO: 121 - DNA, 20 bp, artificial sequence
  chemically synthesized
aatgcatcaagtacaggtcc

SEQ ID NO: 122 - DNA, 22 bp, artificial sequence
  chemically synthesized
ctatgaccagtatgtaccacta

SEQ ID NO: 123 - DNA, 18 bp, artificial sequence
  chemically synthesized
aaccctgcttgtaatgcc

SEQ ID NO: 124 - DNA, 43 bp, artificial sequence
  chemically synthesized
actgttgggtttgaggatctttttggttaacttacctagtggc

SEQ ID NO: 125 - DNA, 42 bp, artificial sequence; identical to SEQ ID NO: 113
  chemically synthesized

SEQ ID NO: 126 - DNA, 22 bp, artificial sequence
  chemically syntesized
ttaagcggattgaagcttgatc

SEQ ID NO: 127 - DNA, 22 bp, artificial sequence
  chemically synthesized
ccaaactgtctttcaacccttt

SEQ ID NO: 128 - DNA, 20 bp, artificial sequence
  chemically synthesized
tgaccagtatgtaccactat

SEQ ID NO: 129 - DNA, 18 bp, artificial sequence
  chemically synthesized
aaccctgcttgtaatgcc

SEQ ID NO: 130 - DNA, 43 bp, artificial sequence
  chemically synthesized
actgttgggtttgaggatcttttggttaacttacctagtggct

SEQ ID NO: 131 - DNA, 42 bp, artificial sequence; identical to SEQ ID NO: 113
  chemically synthesized

SEQ ID NO: 132 - DNA, 22 bp, artificial sequence
  chemically synthesized
ttaagcggattgaagcttgatc

SEQ ID NO: 133 - DNA, 22 bp, artificial sequence
  chemically synthesized
ccaaactgtctttcaacccttt

SEQ ID NO: 134 - DNA, 22 bp, artificial sequence
  chemically synthesized
tgaccagtatgtaccactattg

SEQ ID NO: 135 - DNA, 18 bp, artificial sequence
  chemically synthesized
aaccctgcttgtaatgcc

SEQ ID NO: 136 - DNA, 42 bp, artificial sequence
  chemically synthesized
actgttgggtttgaggatctttgttaacttacctagtggctt

SEQ ID NO: 137 - DNA, 42 bp, artificial sequence; identical to SEQ ID NO: 113
  chemically synthesized

SEQ ID NO: 138 - DNA, 22 bp, artificial sequence
  chemically synthesized
ttaagcggattgaagcttgatc

SEQ ID NO: 139 - DNA, 22 bp, artificial sequence
  chemically synthesized
ttaagcggattgaagcttgatc

SEQ ID NO: 140 - DNA, 20 bp, artificial sequence
  chemically synthesized
gaccagtatgtaccactatt

SEQ ID NO: 141 - DNA, 18 bp, artificial sequence
  chemically synthesized
aaccctgcttgtaatgcc

SEQ ID NO: 142 - DNA, 43 bp, artificial sequence
  chemically synthesized
actgttgggtttgaggatctttggttaacttacctagtggctt

SEQ ID NO: 143 - DNA, 42 bp, artificial sequence; identical to SEQ ID NO: 113
  chemically synthesized

SEQ ID NO: 144 - DNA, 22 bp, artificial sequence
  chemically synthesized
ttaagcggattgaagcttgatc

SEQ ID NO: 145 - DNA, 22 bp, artificial sequence
  chemically synthesized
ccaaactgtctttcaacccttt

SEQ ID NO: 146 - DNA, 19 bp, artificial sequence
  chemically synthesized
cgtgaacgatgaaggtctt

SEQ ID NO: 147 - DNA, 22 bp, artificial sequence
  chemcially synthesized
accacactctagactgatagtt

SEQ ID NO: 148 - DNA, 40 bp, artificial sequence
  chemically synthesized
gcgactgctggcacatagttaagaatgactctagcaggca

SEQ ID NO: 149 - DNA, 42 bp, artificial sequence
  chemically synthesized
acataggtcgcaagcgttatccctgcctttaacaccagactt

SEQ ID NO: 150 - DNA, 20 bp, artificial sequence
  chemically synthesized
gtacagtcaaactccagcca

SEQ ID NO: 151 - DNA, 22 bp, artificial sequence
  chemically synthesized
ggatttattgggcgtaaagcaa

SEQ ID NO: 152 - DNA, 19 bp, artificial sequence
  chemically synthesized
cgtgaacgatgaaggtctt

SEQ ID NO: 153 - DNA, 22 bp, artificial sequence
  chemically synthesized
accacactctagactgatagtt

SEQ ID NO: 154 - DNA, 39 bp, artificial sequence
  chemically synthesized
gcgactgctggcacatagtaatgactctagcaggcaatg

SEQ ID NO: 155 - DNA, 42 bp, artificial sequence; identical to SEQ ID NO: 149
  chemically synthesized

SEQ ID NO: 156 - DNA, 20 bp, artificial sequence
  chemically synthesized
tggtacagtcaaactccagc

SEQ ID NO: 157 - DNA, 22 bp, artificial sequence
  chemically synthesized
ggatttattgggcgtaaagcaa

SEQ ID NO: 158 - DNA, 19 bp, artificial sequence
  chemically synthesized
cgtgaacgatgaaggtctt

SEQ ID NO: 159 - DNA, 22 bp, artificial sequence
  chemically synthesized
accacactctagactgatagtt

SEQ ID NO: 160 - DNA, 42 bp, artificial sequence
  chemically synthesized
gctggcacatagttagtcgtcagaagaatgactctagcaggc

SEQ ID NO: 161 - DNA, 42 bp, artificial sequence; identical to SEQ ID NO: 149
  chemically synthesized

SEQ ID NO: 162 - DNA, 20 bp, artificial sequence
  chemically synthesized
gtacagtcaaactccagcca

SEQ ID NO: 163 - DNA, 22 bp, artificial sequence
  chemically synthesized
ggatttattgggcgtaaagcaa

SEQ ID NO: 164 - DNA, 19 bp, artificial sequence
  chemically synthesized
cgtgaacgatgaaggtctt

SEQ ID NO: 165 - DNA, 22 bp, artificial sequence
  chemically synthesized
accacactctagactgatagtt

SEQ ID NO: 166 - DNA, 41 bp, artificial sequence
  chemically synthesized
gcgactgctggcacatagttagaatgactctagcaggcaat

SEQ ID NO: 167 - DNA, 42 bp, artificial sequence; identical to SEQ ID NO: 149
  chemically synthesized

SEQ ID NO: 168 - DNA, 20 bp, artificial sequence
  chemically synthesized
tggtacagtcaaactccagc

SEQ ID NO: 169 - DNA, 22 bp, artificial sequence
  chemically synthesized
ggatttattgggcgtaaagcaa

SEQ ID NO: 170 - DNA, 20 bp, artificial sequence
  chemically synthesized
cattactgacgcttaggctt

SEQ ID NO: 171 - DNA, 20 bp, artificial sequence
  chemically synthesized
gccaaggatgtcaagtctag

SEQ ID NO: 172 - DNA, 41 bp, artificial sequence
  chemically synthesized
cttcactaccgaagggatcgccctagtagtccacaccgtaa

SEQ ID NO: 173 - DNA, 39 bp, artificial sequence
  chemically synthesized
gcctgggtagtacattcgcaaaacatgctccaccacttg

SEQ ID NO: 174 - DNA, 22 bp, artificial sequence
  chemically synthesized
tccgacagctagtatctatcgt

SEQ ID NO: 175 - DNA, 22 bp, artificial sequence
  chemically synthesized
tgaaactcaaacggaattgacg

SEQ ID NO: 176 - DNA, 19 bp, artificial sequence
  chemically synthesized
cgtgaacgatgaaggtctt

SEQ ID NO: 177 - DNA, 22 bp, artificial sequence
  chemically synthesized
accacactctagactgatagtt

SEQ ID NO: 178 - DNA, 37 bp, artificial sequence
  chemically synthesized
gcgactgctggcacatagtgactctagcaggcaatgg

SEQ ID NO: 179 - DNA, 42 bp, artificial sequence; identical to SEQ ID NO: 149
  chemically synthesized

SEQ ID NO: 180 - DNA, 22 bp, artificial sequence
  chemically synthesized
aaagtggtacagtcaaactcca

SEQ ID NO: 181 - DNA, 22 bp, artificial sequence
  chemically synthesized
ggatttattgggcgtaaagcaa

SEQ ID NO: 182 - DNA, 18 bp, artificial sequence
  chemically synthesized
caagtggtggagcatgtt

SEQ ID NO: 183 - DNA, 19 bp, artificial sequence
  chemically syntehsized
tcccttccttcctccaatt

SEQ ID NO: 184 - DNA, 39 bp, artificial sequence
  chemcially synthesized
cgacaaccatgcaccacctctagacttgacatccttggc

SEQ ID NO: 185 - DNA, 42 bp, artificial sequence
  chemically synthesized
cagctcgtgtcgtgagatgtttaactaacgataagggttgcg

SEQ ID NO: 186 - DNA, 21 bp, artificial sequence
  chemically synthesized
gtcactcggttaacctccatt

SEQ ID NO: 187 - DNA, 18 bp, artificial sequence
  chemically synthesized
ggttaagtcccgcaacga

SEQ ID NO: 188 - DNA, 20 bp, artificial sequence
  chemically synthesized
aatgactctagcaggcaatg

SEQ ID NO: 189 - DNA, 22 bp, artificial sequence
  chemically synthesized
accacactctagactgatagtt

SEQ ID NO: 190 - DNA, 41 bp, artificial sequence
  chemically synthesized
cggataacgcttgcgaccttaagtgacgactaactatgtgc

SEQ ID NO: 191 - DNA, 40 bp, artificial sequence
  chemically synthesized
aagcgcaggcggattgaaccaatgcatacaactgttaagc

SEQ ID NO: 192 - DNA, 19 bp, artificial sequence
  chemically synthesized
tgtattaccgcgactgctg

SEQ ID NO: 193 - DNA, 20 bp, artificial sequence
  chemically synthesized
agtctggtgttaaaggcagc

SEQ ID NO: 194 - DNA, 20 bp, artificial sequence
  chemically synthesized
aatgactctagcaggcaatg

SEQ ID NO: 195 - DNA, 22 bp, artificial sequence
  chemically synthesized
accacactctagactgatagtt

SEQ ID NO: 196 - DNA, 38 bp, artificial sequence
  chemically synthesized
gataacgcttgcgacctaagtgacgactaactatgtgc

SEQ ID NO: 197 - DNA, 40 bp, artificial sequence; identical to SEQ ID NO: 191
  chemically synthesized

SEQ ID NO: 198 - DNA, 19 bp, artificial sequence
  chemically synthesized
tgtattaccgcgactgctg

SEQ ID NO: 199 - DNA, 20 bp, artificial sequence
  chemically synthesized
agtctggtgttaaaggcagc

SEQ ID NO: 200 - DNA, 18 bp, artificial sequence
  chemically sythesized
caagtggtggagcatgtt

SEQ ID NO: 201 - DNA, 20 bp, artificial sequence
  chemically synthesized
gtttgcagccctagacataa

SEQ ID NO: 202 - DNA, 40 bp, artificial sequence
  chemically synthesized
cgacacgagctgacgacaaccttggcaaagttatggaaac

SEQ ID NO: 203 - DNA, 41 bp, artificial sequence
  chemically synthesized
tgggttaagtcccgcaacgccaatttacattagcagtctcg

SEQ ID NO: 204 - DNA, 18 bp, artificial sequence
  chemically synthesized
catgcaccacctgtcact

SEQ ID NO: 205 - DNA, 22 bp, artificial sequence
  chemically synthesized
cgcaacccttatcgttagttac

SEQ ID NO: 206 - DNA, 21 bp, Artificial sequence
  chemically synthesized
cgcataagaactttagttcgc

SEQ ID NO: 207 - DNA, 19 bp, artificial sequence
  chemically synthesized
aagaccttcatcgttcacg

SEQ ID NO: 208 - DNA, 43 bp, artificial sequence
  chemically synthesized
tagctacacgtcattgccttggagggttcgttatttgatgagg

SEQ ID NO: 209 - DNA, 40 bp, artificial sequence
  chemically synthesized
cacaatgggactgagacacggagctttcgctcattgtgaa

SEQ ID NO: 210 - DNA, 22 bp, artificial sequence
  chemically synthesized
cctaccaactagctgatatggc

SEQ ID NO: 211 - DNA, 18 bp, artificial sequence
  chemically synthesized
tactcctacgggaggcag

SEQ ID NO: 212 - DNA, 18 bp, artificial sequence
  chemically synthesized
tgggtgtgaaccatgaga

SEQ ID NO: 213 - DNA, 19 bp, artificial sequence
  chemically synthesized
agtccttccacgataccaa

SEQ ID NO: 214 - DNA, 39 bp, artificial sequence
  chemically synthesized
tccatagggtgccaggctgtatgacaacagcctcaagat

SEQ ID NO: 215 - DNA, 42 bp, artificial sequence
  chemically synthesized
ctttctttgcagcaatgcctccagttgtcatggatgaccttg

SEQ ID NO: 216 - DNA, 19 bp, artificial sequence
  chemically synthesized
ctgccttcctcacctgatg

SEQ ID NO: 217 - DNA, 18 bp, artificial sequence
  chemically synthesized
tgcaccaccaactgctta

SEQ ID NO: 218 - DNA, 16 bp, artificial sequence
  chemically synthesized
ccccaaaggccaggct

SEQ ID NO: 219 - DNA, 19 bp, artificial sequence
  chemically synthesized
agaagggatgggagagagc

SEQ ID NO: 220 - DNA, 42 bp, artificial sequence
  chemcially synthesized
ggaatggggagaagggcaggttaaatgtcaccgggaggattg

SEQ ID NO: 221 - DNA, 41 bp, artificial sequence
  chemically synthesized
cggaaaccagatctcccaccggctacagaaaggtcagcagc

SEQ ID NO: 222 - DNA, 18 bp, artificial sequence
  chemically synthesized
atcaagtggggcgatgct

SEQ ID NO: 223 - DNA, 19 bp, artificial sequence
  chemically synthesized
gggcagagatgatgaccct

SEQ ID NO: 224 - DNA, 39 bp, artificial sequence
  chemically synthesized
gcactcaccccagccttctcgctgagtacgtcgtggagt

SEQ ID NO: 225 - DNA, 40 bp, artificial sequence
  chemcially synthesized
aagctgactcagccctgcaaaccctgcaaatgagcctaca

SEQ ID NO: 226 - DNA, 18 bp, artificial sequence
  chemically synthesized
gttgacccgaccccaaag

SEQ ID NO: 227 - DNA, 18 bp, artificial sequence
  chemically synthesized
aagggatgggagagagcc

SEQ ID NO: 228 - DNA, 39 bp, artificial sequence
  chemically synthesized
cggaatggggagaagggcagatgtcaccgggaggattgg

SEQ ID NO: 229 - DNA, 41 bp, artificial sequence
  chemically synthesized
cggaaaccagatctcccaccgccagctacagaaaggtcagc